(19) United States
(12) Patent Application Publication
(10) Pub. No.: US 2009/0247602 A1
(43) Pub. Date: Oct. 1, 2009

(54) COSMETIC USE OF CONJUGATED COMPOUNDS OF INDOLIC AUXINS

(75) Inventor: Marie-Christine Seguin, Monaco (MC)

Correspondence Address: VOLPE AND KOENIG, P.C., UNITED PLAZA, SUITE 1600, 30 SOUTH 17TH STREET, PHILADELPHIA, PA, 19103, US

(73) Assignee: EXSYMOL S.A.M., Monaco (MC)

(21) Appl. No.: 12/408,376

(22) Filed: Mar. 20, 2009

(30) Foreign Application Priority Data
Mar. 31, 2008 (FR) 08/01755

(51) Int. Cl. A61K 31/405

(52) U.S. Cl. 514/415

(57) ABSTRACT

The invention has for object the cosmetic use and as neurotrophic agent of at least an indolic auxin conjugate with general formula (I):

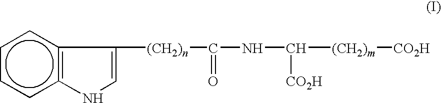

in which:
n is equal to 1, 2 et 3,
and m is equal to 1 et 2.

The invention concerns the selection and use of conjugated compounds of indolic auxins in the cosmetic area, precisely as neurotrophic agent acting on the cutaneous nervous system.

The specialized press recently announced that<<cosmetic is in full green frenezy>>(Cosmétique Magazine, November 2007), thus reporting a market segment with currently growing expansion, the one of natural cosmetics labelled “organic” from a biological and ecological production. In addition, recent alarmist media campaigns on dangers about some ingredients derived from petrochemistry (phtalates, etc) indirectly contribute to increase this consumer's attraction towards the “natural” and products of which the closeness with nature is simple and unequivocal.

The present invention has been developed in this context, and aims at satisfying this increasing demand for cosmetic ingredients, products or preparations having a clearly displayed “naturality”. For that, the applicant chose the option that consists in proposing some “nature-identical” compounds, accurate replica of substances present in the nature but obtained by chemical way.

By choosing the chemical synthesis rather than the extraction, it is obtained a completely pure molecule thus avoiding problems arising from the unavoidable impurities of the extraction of plant material (low activity, toxicity, instability, etc). This approach is particularly appropriate for obtaining natural compounds found in rare or protected plants.

The thought process of the applicant consisted in screening a group of compounds present in the plant kingdom, and in seeking structural elements in order to grant them with some advantageous properties for a cosmetic application.

The applicant's choice falls on indolic auxins which are organic compounds present in all superior plants with seeds. The auxins are involved in mechanisms of proliferation and cellular division of plants (O. Leyser, Current Biology (2001), vol. 11, R728). They are essential to the development of main vegetative organs: root, stem and leaves (Davies P. J., In Plant Hormones Physiology, Biochemistry and Molecular Biology, P. J. Davies ed. Kluwer Academic Publishers, pp. 1-12).

The interest of the applicant turned to this particular class of auxins having an indole ring and an alkanecarboxylic aliphatic chain. The indole ring is indeed an aromatic heterocyclic structure conferring antioxidant properties to the molecule, as established in different scientific articles (Politi V. and al., Adv. Exp. Med. Biol. (1996), vol. 398, pp. 291-298; Le Borgne M. and al., Ann. Pharm. Fr. (2000), vol. 58, pp. 316-320). The indole-3-propionic acid auxin is even announced as one of the strongest natural antioxidants (Karbonik J. and al., J. Cellular Biochem. (2001), vol. 81, pp. 507-513), superior in any case to melatonin and its indole-3-acetic acid homologue (Chyan Y. J. and al., J. Biol. Chem. (1999), vol. 274, pp. 21937-21942).

Moreover, the applicant has limitatively restricted its interest to auxins' conjugates present at the natural state and resulting from the conjugation of the auxin with the alpha-amino function of an aminoacid through an amide-type bond, these conjugates thus having the following general formula:

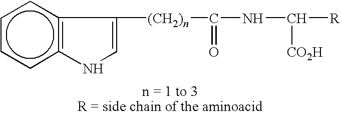

Unlike their non conjugated homologues, these auxins' conjugates have indeed some physico-chemical properties suitable to a cosmetic active ingredient: a water solubility and more generally a polarity that contributes to limit the transcutaneous crossing and the systemic exposure arising from scavenging by microcirculation (resorption).

In the plant, the role of these conjugates is not entirely defined, but it is admitted, in particular for conjugates of indole-3-acetic acid, that they are storage and transport means of the auxin (Woodward A. W. and al., Annals of Botany (2005), vol. 95, pp. 707-735, and cited references). These conjugates maintain an antioxidant power, in particular a protective role against a peroxidative damage (Cohen J. D. and al., Annual Rev. Plant Physiol. (1982), vol. 33, pp. 403-430).

By the way, the antioxidant properties of the conjugated species of indolic auxins have been already used. It is especially to mention the international application WO 2005/062851 which claims a pharmaceutical composition based on amides derivatives of indole-3-propionic acid and useful in the prevention or treatment of conditions associated to an oxidative state.

Chemical replica of a large panel of aminoacid conjugates of indolic auxins were thus obtained by the applicant through some typical synthesis methods, by coupling of the indole-3-acetic acid (“IAA”) or its both homologues, the indole-3-propionic (“IPA”) and indole-3-butyric (“IBA”) acids, to different natural aminoacids chosen amongst of alanine, arginine, aspartic acid, cysteine, glutamic acid, glycine, histidine, leucine, lysine, methionine, phenylalanine, proline, serine, tryptophan, tyrosine and valine.

The systematic screening of properties of each of these synthetized conjugated compounds have been then performed. It has been so confirmed the hereabove-mentioned antioxidant potential, with an important ability for all these conjugated species to inhibit the oxidation of desoxyribose in the tested oxidant system produced by the oxidase xanthin/hypoxanthin pair.

In other respects, and forming the basis of the present invention, it has been discovered for a very small panel of structures limited to the only conjugates of aspartic and glutamic acids, an unexpected property that is a said-neurotrophic activity. This activity is also found in a class of endogenous proteins, the neurotrophins, and in particular in the “NGF” (or “nerve growth factor”), an essential growth and survival factor in the formation and the maintenance of the cutaneous nervous network (M. Bothwell, J. Invest. Dermatol. Symposium Proceedings (1997), vol. 2, pp. 27-30). This remarkable activity, without any connection with antioxidant properties of these compounds, has been evidenced through an in vitro test that consists in measuring the neurites outgrowth kinetics from rat embryos sensory neurons selected for their physiology very similar to the one of human sensory neurons.

Globally, six auxins' conjugates resulting from the coupling of indole-3-acetic, indole-3-propionic and indole-3-butyric acids with successively aspartic and glutamic acids have displayed a significant neurotrophic activity in this test.

Skin being a richly innervated organ by a network of nervous fibres present in the hypodermis, the dermis and up to the epidermis (L. Misery, Int. J. Cometic Sc. (2002), vol. 24, pp. 111-116), the invention has thus for first aspect the cosmetic use and as neurotrophic agent of at least an indolic auxin conjugate with general formula (I):

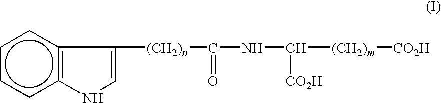

in which:
n is equal to 1, 2 et 3,
and m is equal to 1 et 2.

According to an embodiment of the invention, the conjugate of formula (I) is chosen amongst the indolyl-3-acetylglutamic (n=1 and m=2; “IAA-Glu”) and indolyl-3-butyrylaspartic (n=3 and m=1; “IBA-Asp”) acid compounds, preferentially the indolyl-3-butyrylaspartic acid compound.

Advantageously used in a cosmetic composition, the hereabove-described conjugate of formula (I) is present in such a composition in a quantity comprised between 0.001 and 0.1% % in weight in relation to total weight of the said composition, preferably between 0.01 and 0.05% in weight. This cosmetic composition especially aims at preserving the cutaneous nervous endings.

As it is today clearly admitted the involvement of the cutaneous nervous system in various cutaneous perturbations related to aging, for instance desquamation, cutaneous dryness, altered healing, a decline in the sensorial function, etc (L. Misery, Int. J. Cosmetic Sci. (2002), vol. 24, pp. 111-116), it is particularly selected the use of said indolic auxin conjugates of formula (I) in a cosmetic composition intended for fighting against disorders related to skin aging process, and notably neurodegenerescence process.

According to an other embodiment of the invention, the said composition is susceptible to comprise, furthermore, an antioxidant agent such as vitamins (C, E, and D), ascorbic acid or else phenolic and polyphenolic compounds (flavonoids, etc), this in order to obtain a strengthened neuroprotective effect resulting of the combination of an antioxidant effect and of a neurotrophic effect.

This same composition can also comprise a physiologically compatible excipient with skin. It is adapted to a topical cutaneous administration and presented under all typical forms for such an administration. In an advantageous embodiment, it is formulated under the form for instance of emulsion, cream, milk, gel, lotion, etc.

Finally, according to a second aspect of the invention, the present invention concerns a cosmetic treatment process aiming at preserving the cutaneous nervous endings. This process is used by applying on skin, eventually in a physiologically compatible excipient, a cosmetically efficient quantity of at least an indolic auxin conjugate as previously defined, preferably the indolyl-3-butyrylaspartic acid compound.

The invention is illustrated by the following test hereabove-mentioned in the specification of the invention.

Test: Evidence of the Neurotrophic Activity of Aminoacid Conjugates of Indolic Auxins

The experimental study has been achieved on dissociated sensory neurons from explants of rachidian ganglia of rat embryos, and prepared according to the method described by Hall A. K. and al (J. Neurosci. (1997), vol. 17, pp. 2775-2784). After dissection and dissociation, sensory neurons are at once incubated in the following culture media (in hexaplicate), according to three situations:
case 1: culture medium without NGF, and without active ingredient according to the invention (control)
case 2: culture medium without NGF, with an active ingredient according to the invention
case 3: culture medium with NGF (positive reference)

After an incubation time of 72 hours, cells are washed and fixed. They are then labelled by an anti β-tubulin antibody reacting with cellular bodies and outgrowths of neurons. After a disclosure with an Alexa Fluor 488 secondary antibody, colour picture inputs were proceeded (using “In Cell Analyzer 1000™”). These digital data are then used for measuring a relative density of neurite length compared to the non treated control (case 1).

The neurotrophic effect of tested compounds is expressed by an increase (in μm) of this density of neurite outgrowth of neurons, synonymous with a strengthended development kinetic of neuronal network and a better viability (survival) of this latter. Results are reported in the following table:

| | | Average | |
|---|---|---|---|
| | | density of | |
| | | neurite | |
| | | outgrowth of | |
| | concentration | sensory | % compared to |
| substance | (mg/ml) | neurons (μm) | the control |
| control (case | — | 6284 | — |
| 1) | | | |
| IAA-Glu (case | 0.12 | 7335 | 117 |
| 2) | | | |
| IAA-Asp (case | 0.12 | 6820 | 108 |
| 2) | | | |
| IPA-Glu (case | 0.12 | 8247 | 131 |
| 2) | | | |
| IPA-Asp (case | 0.12 | 6980 | 111 |
| 2) | | | |
| IBA-Glu (case | 0.12 | 7195 | 114 |
| 2) | | | |
| IBA-Asp (case | 0.12 | 8297 | 132 |
| 2) | | | |
| NGF reference | 5.10⁻⁶ | 11750 | 187 |
| (case 3) | | | |

1. A method of preserving cutaneous nervous endings comprising administration of at least an indolic auxin conjugate with general formula (I): 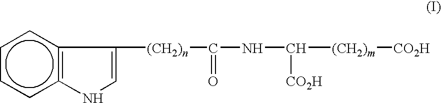
in which:
n is equal to 1, 2 or 3,
and m is equal to 1 or 2,
to skin of a subject.

2. A method of maintaining a cutaneous nervous network comprising topical application of at least an indolic auxin conjugate with general formula (I): 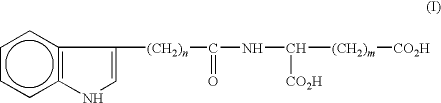
in which:
n is equal to 1, 2 or 3,
and m is equal to 1 or 2,
to skin of a subject.

3. The method according to claim 1, wherein said conjugate is selected from the group consisting of indolyl-3-acetylglutamic and indolyl-3-butyrylaspartic acid compounds.

4. The method according to claim 3, wherein said conjugate is the indolyl-3-butyrylaspartic acid compound.

5. The method according to claim 1, wherein said conjugate is present in a quantity between 0.001 and 0.1% in weight of a cosmetic composition in relation to total weight of said composition.

6. The method according to claim 5, wherein said administration is applied to affect disorders related to skin aging.

7. The method according to claim 1, wherein said composition further comprises an antioxidant.

8. The method according to claim 7, wherein said antioxidant is selected from the group consisting of vitamins, ascorbic acid, phenolic compounds and polyphenolic compounds.

9. The method according to claim 5, wherein said composition comprises a physiologically compatible excipient with skin that is adapted to topical administration.

10. A cosmetic treatment process intended for preserving cutaneous nervous endings comprising applying on skin a cosmetically efficient quantity of at least an indolic auxin conjugate with general formula (I): 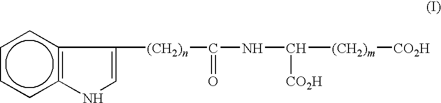
in which:
n is equal to 1, 2 or 3,
and m is equal to 1 or 2.

11. The method according to claim 2, wherein said conjugate is selected from the group consisting of indolyl-3-acetylglutamic and indolyl-3-butyrylaspartic acid compounds.

12. The method according to claim 11, wherein said conjugate is the indolyl-3-butyrylaspartic acid compound.

13. The method according to claim 2, wherein said conjugate is present in a quantity between 0.001 and 0.1% in weight of a cosmetic composition in relation to total weight of said composition.

14. The method according to claim 13, wherein said topical application is applied to affect disorders related to skin aging.

15. The method according to claim 2, wherein said composition further comprises an antioxidant.

16. The method according to claim 15, wherein said antioxidant is selected from the group consisting of vitamins, ascorbic acid, phenolic compounds and polyphenolic compounds.

17. The method according to claim 15, wherein said composition comprises a physiologically compatible excipient with skin that is adapted to topical administration.
